(19) United States
(12) Patent Application Publication
(10) Pub. No.: US 2008/0085525 A1
(43) Pub. Date: Apr. 10, 2008

(54) Chromatographic Analysis Devices And Test Kits

(75) Inventor: Johannes Robertus Van Herwijnen, Linschoten (NL)

Correspondence Address: TROUTMAN SANDERS LLP, 600 PEACHTREE STREET , NE, ATLANTA, GA, 30308, US

(73) Assignee: Teststrip B.V., Oud-Beijerland (NL)

(21) Appl. No.: 11/721,758

(22) PCT Filed: Dec. 14, 2005
(86) PCT No.: PCT/NL05/00861

§ 371 Date: Jun. 14, 2007

(30) Foreign Application Priority Data
Dec. 14, 2004 (NL) 1027737

(51) Int. Cl. G01N 33/566; C07K 14/00; G01N 33/558

(52) U.S. Cl. 435/7.25; 436/514; 530/402

(57) ABSTRACT

Chromatographic analysis devices (1) are described for the detection of blood parameters, consisting of cell components of cells circulating in blood of human or animal origin, or of plasma components, in a sample of a bodily fluid containing such cells, comprising a strip-shaped chromatographic medium (3) having a sample application zone (4), a suction zone (5), and at least one detection zone (6, 7) wherein application zone, suction zone and detection zone are mutually separated from each other by at least one separation zone (8a, 8b, 8c), which is (are) each provided with a material inhibiting lysis of the circulation cells. Test kits for performing such analysis are described as well.

The invention relates to chromatographic analysis devices for the detection of blood parameters, consisting of cell components of cells circulating in blood of human or animal origin, or of plasma component(s) in blood of human or animal origin, in a sample of a bodily fluid containing such cells, comprising a strip-like chromatographic medium having a sample application zone, a suction zone, and at least one detection zone, wherein application zone, suction zone and detection zone(s) are always separated from each other by at least one separation zone.

Such an analysis device is used for the fast diagnosis or therapeutic check of a number of disorders. In this context, the chromatographic medium is more particularly a so-called thin layer system, wherein a liquid containing the bodily liquid to be analysed, runs over the absorbing medium while performing the test.

In the case of cell components of erythrocytes, the detection usually took place on the basis of agglutination. However, such a determination proved to be rather inaccurate due to contaminations present in the erythrocytes containing sample. The detection of early IgM antibodies usually also took place on the basis of agglutination and/or ELISA techniques. However, such a determination did not prove to be usable under non-laboratory conditions. Since for an analysis usually only an amount of 3-50 μl of the sample to be analysed is used, a high detection sensitivity of the analysis device is of high importance. Besides, there is an increasing need to be able to detect, in a very simple way, directly, i.e. within several minutes, for example erythrocytes of human or animal origin, in order to thus determine the blood group, or to be able to detect the (early) antibodies of human or animal origin, which are made in an early stage of an immune reaction (so called early antibodies), and thus to be able to determine a possible disease in an initial phase.

In the human field, such a determination is especially important in blood transfusions, for example on an intensive care department of a hospital, and in the “doctor's office”, and also for early determination of human pathogenic infections, such as for example with the Epstein Barr virus (Pfeiffer's disease), toxoplasma, the tubercle bacillus, the West Nile virus, the Cytomegalovirus. In the veterinary field, the detection of the blood group is also important, such as for example with cats, where it can help preventing the occurrence of the “Feline Neonatal Erythrolysis Syndrome” (by determining the blood groups of possible parents). This syndrome is namely caused by non-corresponding blood groups with cats, and results in lysis of the red blood cells of the cat directly after birth. Besides, a so-called haemolytic transfusion reaction can occur during or after a blood transfusion, wherein also lysis of the red blood cells occurs due to incompatible blood groups.

Now, a chromatographic analysis device has been found for the detection of blood parameters, consisting of cell components of cells circulating in blood of human or animal origin, or plasma components) in blood of human or animal origin, which enables a fast detection, and moreover has a large sensitivity.

More particularly, it has been found that when lysis of the cells circulating in blood or bodily fluid is prevented, this enables a fast and reliable test.

The chromatographic analysis device of the type mentioned in the preamble is therefore characterized in that the separation zone(s) is (are) provided with a material preventing lysis of the circulating cells.

On the basis of this principle it proved to be possible to develop analysis devices wherein erythrocytes, as such, are used as markers, but also analysis devices wherein labelled (with gold) antibodies can be used as a marker, as will be explained below in more detail.

In a preferred embodiment, the material preventing lysis of the circulating cells consists of a dispersant, in particularly a poloxamer.

According to another preferred embodiment, the material preventing lysis of circulating cells consists of a material aggregating circulating cells, in particular a hydrocarbon, such as for example lactose.

The test sample to be analysed with the present device, in a first embodiment, is in particular complete blood. However, it can also be used for the analysis of other bodily liquids which contain or may contain (blood)cells, such as urine, cerebrospinal liquid and the like.

Below, in a first variation, the invention is explained, with the detection of cell components of erythrocytes (=red blood cells). However, the invention is not limited thereto. Also other cells which have ended up in the circulation, can be detected. The selection therefore takes place on the basis of cell surface markers, such as for example adhesion molecules (EP-CAM, N-CAM (=CD56), other CD markers (I-CAM, PECAM, H-CAM), DAF, Fibronectine receptor, Laminine receptor, etc.), but also other cell surface molecules such as for examples mucines (MUC1, MUC2, MUC3, etc.), particular CD markers, tumor markers (CEA, CA15.3, CA125, CA19.9, AFP, PSA, BRCA1, BRCA2, etc.), growth or growth-inhibiting factor receptors (TGF, EGF, TNF, INF, FGF, “Insulin like growfactor”, etc.), antibodies (myelomas), MHC molecules, particular viral cell surface antigens (Flu viruses, SARS, HIV, HPV, EBV), specific glycosylation patterns (T, Tn, Sialyl-Tn, etc.), T-cell receptors present and coming to expression on lymphoid cells after infection or contact with infectious organisms such as viruses (SARS, HPV, EBV etc.) or bacterial organisms (TBC, Toxoplasma, E. cuniculi etc.) or parasitic organisms (Leismania, Schistosomia, malaria etc.), further intracellular elements which are detectable in case of damage of the cell, such as intermediate filaments (keratins, such as for example keratin 19), vimentin, desmin, neurofilaments, “glial fibrillaric acid”) which, all by means of monoclonal/polyclonal antibodies, or lectins, are selected and detected.

Therefore, in the present invention it is important that the cells to be detected remain intact during the detection itself; only a surface property of the cell is utilised for the detection.

Erythrocytes or red blood cells have an external membrane containing hydrocarbon groups. These hydrocarbons or sugar groups are specific for the immunological properties of the red blood cell concerned, and form the basis of the classification of red blood cells in blood groups.

Because the red blood cell is left intact during the test, it is possible, in a first embodiment variation, by a suitable choice of reagents in the detection zone(s), to make binding of the various cell components directly visible.

To that end, according to the first embodiment variation, the detection zone of the chromatographic analysis device according to the invention is provided with a lectin or an anti-blood cell antibody containing material.

Due to the fact that the erythrocytes are left intact, they can hereby be used themselves as an indicator in the detection zone.

It is noted that lectins consist of proteins which bind specifically and non-covalently to sugar groups being present on the surface of blood cells.

Although there are many different kinds of lectins, the lectin being present in the detection zone of the present analysis device is preferably blood cell group specific.

Examples thereof are concanavalin A, Helix aspersa lectin, Gambucus nigra lectin, and Triticum vulgaris lectin.

It is noted that the lectin or the anti-blood cell antibody may be bound non-covalently to the chromatographic medium, or cross-linked covalently to it. Such procedures are commonly known in the technical field.

Besides, the material used for the chromatographic medium may consist of a woven or non-woven fabric, paper, cellulose, glass fibre, polyester or another polymeric material, nitrocellulose or a combination thereof, but is preferably polyester.

For improving the detecting power of the detection zone, this zone is preferably provided with a salt with a bivalent transition metal. Chlorides of calcium, manganese and magnesium proved to provide excellent effects for this, especially in the presence of a lectin.

The invention further relates to a test kit, for the detection of cell components of erythrocytes circulating in blood of human or animal origin, comprising a chromatographic device as described above, a sample container provided with an aqueous erythrocytes stabilising solution provided with a lysis of erythrocytes preventing material, and, if desired, a container with dilution liquid consisting of an aqueous solution having a pH in the physiological pH-range, and provided with a lysis of erythrocytes preventing material.

Preferred embodiments of such a test kit are shown in the claims 11-14.

According to a another embodiment of the invention, it is possible, by a suitable choice of reagents in the application zone, to stop the red blood cells, while plasma components (such as antibodies: IgM, IgG, “acute phase proteins”, other proteins) can go on, and be made visible in the detection zone(s). This can take place, for example, by treating the haemoglobin with the enzyme thrombin, or by using the (below mentioned Pluronic) buffer with EDTA, in the presence or absence of a sugar, in order to bind and stop the blood cells, whereby one can let the plasma components go over the device.

According to this second embodiment variation of the device according to the invention, to that end, the detection zone(s) of the chromatographic analysis device is (are) provided with an antibody directed against a particular plasma component to be detected, such as for example an anti-human IgM antibody.

Such an antibody may be bound to the chromatographic medium, or covalently crosslinked to it.

For the chromatographic medium of course the same materials can be used as mentioned above; in the second variation, however, nitrocellulose proved to provide the best result, for the time being.

For visualising a seeked plasma component, which is to be detected, a labelling zone is provided in the second embodiment, which zone contains a label suitable for the component to be detected, chosen from gold labels, carbon labels, metal or non-metal labels (such as based on silica, sulphur, silver, iron), fluorescent, bio-luminescent, chemo-luminescent labels, or latex, preferably a gold label.

This labelling zone, in the second embodiment variation of the invention, is connected to the sample application zone. Preferably, the labelling zone is provided with a labelled recombinant protein or labelled synthetic peptide. In using the device for the detection of EBV-infection, the labelling zone is preferably provided with gold labelled EBV protein, or for example a gold labelled antibody, such as a (anti-)human monoclonal antibody.

In this second embodiment variation of the analysis device according to the invention, the detection zone(s) is (are) preferably provided with an antibody directed against the plasma component to be detected, preferably an anti-human IgM antibody.

Expediently, a film of a bi-functional cross-linking agent is present between the antibody containing zone and the chromatographic medium, in particular a polyethylene glycol, or glutaraldehyde, or a composition containing such group(s).

The chromatographic medium, in the second embodiment variation, is expediently provided with a control zone. This control zone contains, preferably, an antibody or antigen, which can bind the labelled protein or peptide made by labelling and to be detected, while forming a detectable signal. In this case, for example, in using a mouse-monoclonal antibody, the control zone will contain an anti-mouse-monoclonal antibody, etc.

The invention further relates to a test kit for the detection of plasma components in blood of human or animal origin, comprising a chromatographic analysis device as described above, a sample container provided with an aqueous, erythrocytes-stabilizing solution and, if desired, a container with a dilution liquid, consisting of an aqueous solution having an pH in the physiological pH-range, preferably consisting of a phosphate or borate buffer having a molarity of 0.05-2, preferably 0.09-1.3, particularly 0.09-0.9.

It will be clear that the said containers will consist of vials, or tubes, since for the test only small amounts are required.

As is known, the physiological pH-range of most bodily liquids varies between 6.5 and 9.0.

Expedient embodiments of the test kit for the detection of plasma components are shown in the claims 23-27.

In an attractive embodiment variation, however, it is possible to use the chromatographic analysis device according to the first embodiment for the analysis of plasma components. For such an analysis, the test kit is then, in addition to a sample container with an aqueous, erythrocytes-stabilizing solution, and, if desired, a container with a dilution liquid (or dilution buffer) consisting of an aqueous solution having a pH in the physiological pH-range, and a lysis of erythrocytes preventing material, also provided with a container having an indicator for the plasma component to be analysed, as well as, if desired, a container with a so called chase buffer consisting of an aqueous solution having a pH in the physiological pH-range.

The indicator for the plasma component preferably consists of a gold labelled antibody, gold labelled recombinant protein or gold labelled synthetic peptide, wherein this protein or peptide preferably corresponds to the protein or peptide to be analysed.

When using the test kit for the analysis of an EBV infection, the indicator will preferably consist of gold labelled EBV protein. Of course, also different labels can be used, such as those mentioned above.

The indicator for a plasma component is preferably included in an aqueous (buffer) solution having a pH in the physiological pH-range. The container can then for example be a (known) dropper.

However, when the indicator is present as a powder, then a container with a (chase) buffer is further present, in order to secure that, after the indicator is applied to the sample application zone, sufficient contact is possible between indicator and the sample to be analysed.

The gold labelled EBV protein, to be used for an EBV infection, is preferably present in powder form, more preferably as lyophilized powder. Such a gold-protein conjugate forms part of the invention.

The invention will hereinafter be further explained with reference to the accompanying drawing, in which

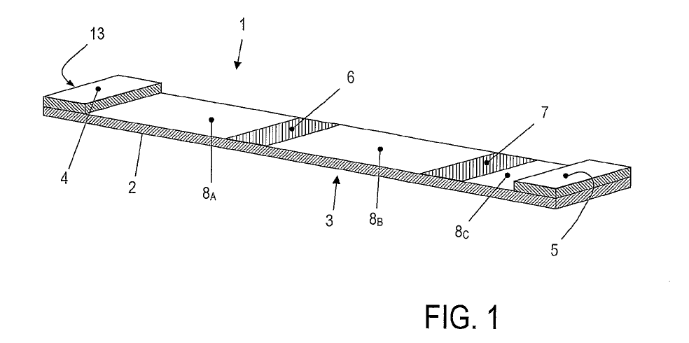

FIG. 1 schematically shows the structure of a first embodiment of an analysis device according to the invention,

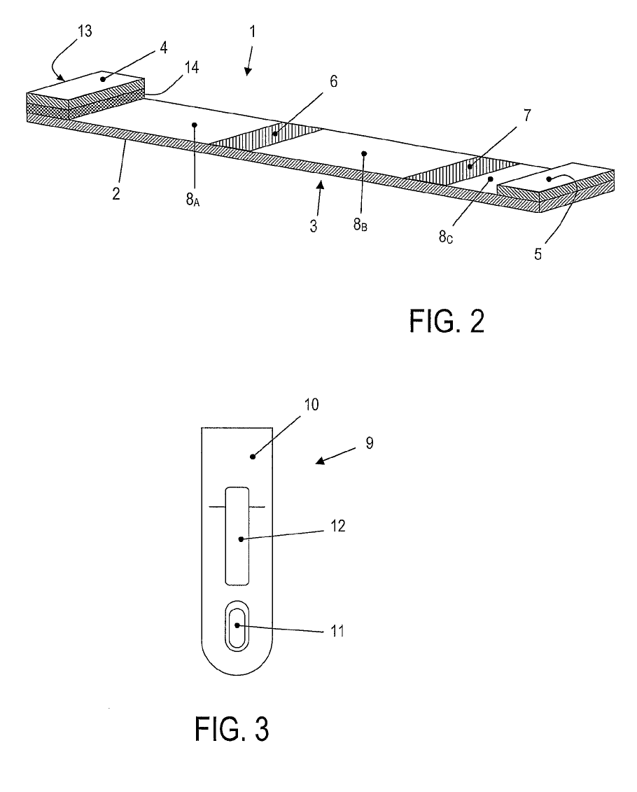

FIG. 2 schematically shows the structure of a second embodiment of an analysis device according to the invention, and

FIG. 3 schematically shows a top view of a container in which an analysis device according to the invention is accommodated.

In FIG. 1, a chromatographic analysis device (1) for the detection of cell components of erythrocytes of human or animal origin is schematically shown. The detection device (1) more particularly consists of an elongated flat strip (3) of material which is suitable as medium for thin-layer chromatography, such as nitrocellulose, cellulose acetate, nylon, rayon, polyester or paper, in particular a polyester. In order to prevent loss of test material during a test and for support, the detection device (1) is preferably provided with a liquid impermeable back layer (2) on the backside of the device. This back layer (2) may consist of for example polystyrene with a glue-like intermediate layer.

The ends of the strip-shaped test device (1) are provided with an application zone (4) and suction zone (5), respectively. Both zones can be formed from any absorbing material that is usually used for this purpose, such as for example filter paper. The size and shape of the zones are chosen depending on the volume of the liquid used in the analysis.

As is known, in a chromatographic test device, liquid is sucked in by capillary action. For obtaining a good reproducible test result, it proved to be expedient to provide the application zone (4) with flow delaying means, for example in the form of channels which are located substantially transverse to the longitudinal direction of the test device (1). The material of the application zone (4) and suction zone (5) may be the same as of strip (3). Preferably, application zone and suction zone consist of a thickened part of strip (3); however, this is not required.

It is noted that the test device can have for example a length of 3-8 cm, and a width of 2-8.5 mm; however, it will be clear that other dimensions may also be used.

In order to be able to detect cell components of erythrocytes with the test device (1), the device is provided with one or more detection zones (6, 7), each containing a detection means being specific for one particular cell component. This detection means is applied in a way known per se, such as by impregnating, pressing or spraying a solution of the detection means in a suitable solvent, such as water, followed by removal of the solvent.

The detection means for detecting cell components of erythrocytes is in particular a lectin or an anti-blood cell antibody.

Lectins are proteins which are produced by plants and some animals, and specifically and non-covalently bind to sugar groups, such as those present on the surface of blood cells.

In particular, however not limited thereto, the lectin is: concanavalin A, abrine, limuline, or one of the lectins which are produced by Agaricus bisporus, Anguilla anguilla, Arachis hypogaea, Bandeiraea simplicifolia, Bauhinia purpurea, Caragana arborescens, Cicer arietinum, Codium fragile, Datura stramoniumn, Dolichos biflorus, Erythrina corallodendron, Erythrina cristagalli, Euonymnus europaeus, Glycine max, Helix aspersa, Helix pomatia, Lathyrus odoratus, Lens culinaris, Licopersicon esculentum, Maclura pomifera, Momordica charantia, Micoplasma gallisepticum, Naja mocambigue, Naja kaouthia, Perseun americana, Phaseolus coccineus, Phaseolus limesis, Phaseolus vulgaris, Phytolacca americana, Pisum sativum, Pseudomonas aeruginosa, Psophocarpus tetragonolobus, Ptilota plumosa, Ricinus communis, Robinia pseudoacacia, Sambucus nigra, Solanum tuberosum, Sophora japonica, Tetragonolobus purpureas, Triticum vulgaris, Ulex europaeus, Vicia faba, Vicia sativa, Vicia villosa, Vigna radiata, Viscum album en Wisteria floribunda.

In the case of blood group determination of cats, the lectin is preferably chosen from concanavalin A, or the lectin which is produced by Helix aspersa, Sambucus nigra, or Triticum vulgaris.

When the detection means is an antibody, both a monoclonal and polyclonal antibody may be used. The choice of a particular antibody is known to the person skilled in the art and therefore does not need to be explained in more detail.

It is noted that the analysis device according to the invention is preferably also provided with a control detection zone (not shown) containing a combination of the detection means of the various detection zones, or preferably a lectin or antibody that binds to all cell components to be detected in the analysis device (1). Because these components have to be made visible with an indicator while analysing plasma components, in that case, it is recommended to provide the analysis device with a control detection zone.

When using a lectin such as for example concanavalin A, the detection zones (6, 7) are preferably also treated with a solution of one or more salts with one or more bivalent transition metal ions, in particular MgCl₂, MnCl₂ and/or CaCl₂.

Alternatively, the detection zone may be impregnated with a hydrocarbon that can agglomerate erythrocytes. Such a hydrocarbon is for example mannitol, sorbitol, glucose, lactose, maltose or saccharose. Preferably, lactose is used.

Because the blood cells themselves can function as indicator in the analysis device according to this variation of the invention, a separate indicator in the detection zones and/or control zones is not required.

In order to improve the adherence of the detection means, such as a lectin or an antibody, to the chromatographic material of layer (3) of the present analysis device, before the detection means is applied at the location of the detection and possible control zone, layer (3) may be treated with a solution or suspension of a bi-functional cross-linking agent, followed by removal of the solvent according to a way known per se, while leaving behind a film of a bi-functional cross-linking agent. Examples of suitable bi-functional cross-linking agents are polyethylene glycols with a molecular weight of 500-20,000, and hetero bi-functional cross-linking agents such as glutaraldehyde, MBS, SATA, or ETA, dicarbodiimide etc.

The separation zones (8a, 8b and 8c) are provided with a lysis of erythrocytes inhibiting material. Such a material preferably consists of a dispersant, in particular a poloxamere, such as Pluronic or Tetronic (trademarks of BASF), or an erythrocytes-aggregating material, such as a hydrocarbon, preferably a lactose.

In FIG. 2, a chromatographic analysis device (1) for the detection of first phase (early phase) antibodies according to a second variation of the invention is schematically shown.

This variation relates in particular to a so called “Mu (IgM) capture test” for the detection of early antibodies in blood which is made incoagulable, which is in particular suitable for early, and very fast, detection of for example EBV, CMV, West Nile virus, influenza virus, and other infections.

For such a detection, one or more detection zones (6, 7) is (are), if desired, provided with a detection means being specific for a particular infection. This detection means is applied according to a way known per se.

The detection means for the detection of early IgM anti material, is in particular an anti IgM antibody, which may be both a monoclonal and polyclonal antibody.

In order to improve the adherence of the detection means, i.e. the antibody, to the chromatographic material of layer (3) of the present analysis device, before the detection means is applied at the location of the detection and possible control zone, layer (3) may be treated with a solution or suspension of a bi-functional cross-linking agent, followed by removal of the solvent according to a way known per se, while leaving behind a film of a bi-functional cross-linking agent. Examples of suitable bi-functional cross-linking agents are polyethylene glycols with a molecular weight of 3,000-20,000, and hetero bi-functional cross-linking agents such as glutar-aldehyde, MBS, SATA, or ETA, dicarbodiimide.

As is indicated in FIG. 2, at the sample application zone (4), the present analysis device is also provided with a labelling zone (14); this zone may be located below and/or next to the sample application zone (4).

For the detection of for example early IgM anti-materials, it has proven to be expedient to use an indicator based on gold. To that end, the labelling zone (14) is provided with a gold labelled recombinant protein or gold labelled synthetic peptide, for example a gold labelled EBV protein.

The unique thing of this concept is that specific anti-materials are “fished” from a pool of IgG, IgA, IgM, and IgE, namely, in the present case IgM antibodies. These antibodies can react with the gold from the labelling zone (14), and in the detection zone, with the detection means, such as a suitable antibody, give a colour reaction: gold labels give a deep purple-red colour.

It is noted that in standard concepts, the IgG, IgA and IgE, before applying to a sample to be tested, first has to be removed from it. The device according to the invention overcomes this problem, whereby the detection can be performed much faster and cheaper.

Instead of an indicator based on gold, it is of course possible to use an indicator based on for example carbon or latex, as is mentioned before.

As regards to function and composition, the remaining parts shown in FIG. 2 correspond to those from FIG. 1.

An analysis such as the one that can be performed with a device according to FIG. 2, however, can also be performed with a device according to FIG. 1. Of course, it is then necessary that the indicator is applied to the sample application zone before (or at the same time with or almost at the same time with) the sample to be analysed is applied.

In FIG. 3, a container (9) known per se consisting of two parts fitting together in a clamping way, is shown schematically in top view, for a chromatographic analysis device according to the invention. In this container, the analysis device is situated. The top side (10) of the container (9) is provided with opening (11), beneath which the application zone (4) of the analysis device is situated. Further, the top side of container (9) is provided with an opening (12) which has dimensions such that the detection zones and possible control zone present can be visible. The backside of container (9) which is not provided with openings, is provided on the inside with means for positioning the analysis device according to the invention (not shown). Topside and backside of container (9) are connected to each other in a clamping way.

As is indicated above, for the detection of early antibodies in blood (which is made incoagulable), use can be made of a gold protein label. Such a label can be prepared as follows.

First, a gold sol was produced by boiling gold particles in water or buffer with low ion strength, while using citrate, sugars, or lauric acid, with or without special binding groups such as amino or aldehyde groups, etc., as cores to let the gold particles grow. After cooling and sieving (filtration or centrifugation), this gold sol is used to couple to proteins or peptides, possibly with a hetero functional cross-linking agent, such as glutaraldehyde, at a pH of 5-11, preferably a pH of 6-9. To that end, a protein or peptide is reacted with an excess of glutaraldehyde, the excess of glutaraldehyde is removed after that and subsequently, the protein or peptide glutaraldehyde product is reacted with the gold sol. It is noted that as peptide (or recombinant protein), the peptide (or protein) is used of which the antibody has to be detected in the test device.

After blocking the non-coupled gold particles, with preferably proteins such as BSA, albumin, or PVP or PVA, in a concentration of 1-3%, the coupled gold particles can be purified by centrifuging with high speed and under application of a density gradient. Subsequently, a sugar, such as lactose, glucose, dextran or mannose, protein or surfactant is added to the product achieved as stabiliser, after which the thus obtained conjugate of gold with protein (or peptide) can be used in the present chromatographic analysis device to visualise positive reactions.

The thus formed conjugate can, according to a way known per se, be dried to obtain a powder, or even be lyophilized to obtain a lyophilized powder.

For use in the device according to the invention, the conjugate is preferably included in a buffer solution (such as a salt solution buffered with phosphate with pH of 5-9, preferably approximately 7.4) and subjected to vortexing to obtain a uniform particle dimension distribution of the conjugate particles in the solution. Standardisation of the conjugate solution or suspension, respectively, is preferred therein; in the case of the gold EBV protein conjugate mentioned before, this standardisation can take place by measuring the permeability at 540 nm. Anyway, it is recommended, after applying the conjugate solution and/or after applying the sample to be analysed, to apply a small amount of a chase buffer on the sample application zone, wherein this chase buffer preferably consists of Pluronic, EDTA, bromeline and PBS, preferably with the concentrations indicated below.

The invention is further explained with reference to the following, non-limiting exemplary embodiment.

A chromatographic analysis device according to FIG. 1, consisting of a polyester layer (3), and provided with a polystyrene back layer (2), with a length of approximately 6 cm, width of approximately 0.5 cm and thickness of approximately 0.1 cm, was provided with a sample application zone (4) and suction zone (5), respectively, at the ends at the side directed away from the back layer. Polyester or glass fibre was used as material for both zones, on the understanding that for the sample application zone (4), the channels and fibres, respectively, of the material extend substantially parallel to the short sides (13) of the device (1).

In order to make this analysis device suitable for the determination of 2 blood groups, detection zones (6) and (7) were each provided with a lectin specific for a blood group, for example zone (6): concanavalin A, and zone (7): the lectin produced by Triticum vulgaris, which are specific for blood group A and blood group B, respectively, with cats. Beforehand, these zones are preferably also provided with alfa-lactose and calcium chloride and/or magnesium chloride, as is explained before.

Subsequently, the separation zones (8a, 8b and 8c), and preferably also the detection zone(s), were treated with a block buffer, in order to provide these zones with a lysis of erythrocytes inhibiting material, in particular a poloxamer, such as Pluronic. More particularly, the block buffer consisted of an aqueous solution with 0.3-9 weight percent, in particular 0.5-5 weight percent, expediently 1-2 weight percent, preferably 1 weight percent, of Pluronic, and 0.1-5 weight percent, in particular 0.2-1 weight percent, preferably 0.5 weight percent, of bovine serum albumin. After applying the block buffer solution, for example by spraying, the analysis device (1) was dried for obtaining the analysis device ready for use.

For performing the detection, blood or a blood containing bodily liquid was included in an aqueous buffer solution, consisting of an anticoagulating agent, such a EDTA, preferably 5-15 weight percent, in particular approximately 11 weight percent of a 500 mM solution; a protein digesting enzyme, preferably bromalin, in a concentration of 0.01-9 weight percent, in particular 0.05-6 weight percent, expediently 0.05-4 weight percent, preferably 2 weight percent. Pluronic (BASF PE6400) as lysis inhibiting material in a concentration of 0.2-9 weight percent, in particular 0.05-5 weight percent, preferably 0.3-5 weight percent, in particular 1-1.9 weight percent, more preferably approximately 1.9 weight percent; as well as approximately 0.01 M phosphate buffered salt solution for obtaining a pH of 5-9, in particular 6-8, preferably approximately 7.4. If desired, the buffer solution may contain a small amount of a PEG, preferably PEG 6000, and possibly a small amount of emulsifier, preferably TWEEN 20. In practise, a (dilution) buffer solution consisting of 1.9 weight percent of Pluronic, 11 weight percent of a 500 mM EDTA solution, and 4 weight percent of bromelin, in a solution buffered at pH 7.4 with 0.01 M phosphate, proved to provide excellent results.

It is noted that, because the present analysis is based on the detection of sugar groups on erythrocytes, it proved in practise that bromelin allowed for a clearer detection, possibly by making particular sugar groups accessible which are otherwise not or incompletely accessible.

Alternatively, the blood sample may be included in a aqueous buffer solution consisting of a preservative (such as sodium azide), α-lactose and sodium biphosphate, with a pH of 5-9, in particular 6-8, wherein the concentration of α-lactose amounts to 0.3-6%, in particular 0.5-5%, preferably 2.5%. For improving the detecting power of the device, a combination of α-lactose and Pluronic can be used, wherein the total concentration of both materials is 0.3-6%, in particular 0.5-5%, preferably 2.5%. The sodium azide concentration is expediently approximately 0.005%.

After an amount of blood sample containing, aqueous buffer solution of 50-200 ml, preferably ±60 μl is applied to the sample application zone (4) of the chromatographic analysis device according to the invention, possibly followed by 2×60 μl dilution buffer (as described before), the buffer solution will spread over the various zones and will be sucked by suction zone (5). When blood group A is present, a red band will emerge at the position of detection zone (6), and when blood group B is present, a red band will emerge at detection zone (7), due to the fact that erythrocytes can be utilised as indicator.

If desired, a dilution buffer can be used with the same composition as above-mentioned aqueous buffer solution, however, of course without a protein digesting enzyme.

As is explained above, the present chromatographic analysis device can be used for the blood group determination in the human and veterinarian field. Depending on the number of blood groups, and the exact blood group one wishes to detect, the number of detection zones of the analysis device will be adapted accordingly, and each be provided with a detection means suitable for a blood group. However, such a modification of the device is within the scope of a person skilled in the art and will not be explained in more detail.

1. Chromatographic analysis device for the detection of blood parameters, consisting of cell components of cells circulating in blood of human or animal origin, in a sample of a bodily liquid comprising such cells, or of plasma components in blood of human or animal origin, comprising a strip-like chromatographic medium having a sample application zone, a suction zone, and at least one detection zone wherein application zone, suction zone and detection zones are mutually always separated from each other by at least one separation zone wherein the separation zones is (are) provided with a material inhibiting lysis of the circulating cells.

2. Chromatographic analysis device according to claim 1, wherein the material preventing lysis of circulating cells is a dispersant.

3. Chromatographic analysis device according to claim 1, wherein the material inhibiting lysis of circulating cells consists of circulating cells aggregating material.

4. Chromatographic analysis device according to claim 1, wherein the cells circulating in blood consist of erythrocytes.

5. Chromatographic analysis device according to claim 1, wherein the detection zone, for the detection of cell components of erythrocytes, is provided with a material comprising a lectin or an anti-blood cell antibody.

6. Chromatographic analysis device according to claim 5, wherein the lectin is chosen from concanavalin A, Helix aspersa lectin, Sambucus nigra lectin and Triticum vulgaris lectin.

7. Chromatographic analysis device according to claim 5, wherein the detection zone further contains at least one bivalent transition metal salt.

8. Chromatographic analysis device according to claim 5, wherein between the film containing the lectin or anti-blood cell antibody and the chromatographic medium, a film of a bi-functional cross-linking agent is present.

9. Chromatographic analysis device according to claim 1, wherein the strip-shaped chromatographic medium consists of a woven or non-woven fabric, paper, cellulose, glass fibre, polyester, or nitro cellulose.

10. Test kit for the detection of cell components of erythrocytes circulating in blood of human or animal origin, comprising a chromatographic device as described in claim 1, a sample holder provided with an aqueous, erythrocytes stabilising solution provided with a material inhibiting lysis of erythrocytes, and, if desired, a container with dilution liquid consisting of an aqueous solution with a pH in the physiological pH range, and provided with a material inhibiting lysis of erythrocytes.

11. Test kit according to claim 10, wherein the material inhibiting lysis of erythrocytes consists of erythrocytes aggregating material.

12. Test kit according to claim 10, wherein the material inhibiting lysis of erythrocytes is present in a concentration of 0.2-9 weight percent.

13. Test kit according to claim 10, wherein the erythrocytes stabilising solution further contains a protein digesting enzyme.

14. Test kit according to claim 10, wherein the erythrocytes stabilising solution further contains a preservative.

15. Chromatographic analysis device for the detection of plasma components according to claim 1, wherein the sample application zone is connected to a labelling zone.

16. Chromatographic analysis device according to claim 15, wherein the labelling zone is provided with a labelled recombinant protein or synthetic peptide.

17. Chromatographic analysis device according to claim 16, wherein the detection zones is (are) provided with an antibody directed against a plasma component to be detected.

18. Chromatographic analysis device according to claim 15, wherein, between the antibody containing zone and the chromatographic medium, a film of a bi-functional cross-linking agent is present.

19. Chromatographic analysis device according to claim 18, wherein the chromatographic medium is further provided with a control zone.

20. Chromatographic analysis device according to claim 19, wherein the control zone is provided with an antibody or antigen, which can bind the labelled protein or peptide to be detected, formed by labelling, while forming a detectable signal.

21. Chromatographic analysis device according to claim 20, wherein the strip-shaped chromatographic medium consists of a woven or non-woven fabric, paper, cellulose, glass fibre, polyester, or nitrocellulose, preferably nitrocellulose.

22. Test kit for the detection of plasma components in blood of human or animal origin, comprising a chromatographic device as described in claim 1, a sample container provided with an aqueous, erythrocytes stabilising solution and, if desired, a container with a dilution liquid, consisting of an aqueous solution with a pH in the physiological pH-range.

23. Test kit according to claim 22, wherein the dilution liquid and/or the erythrocytes stabilising solution are provided with a material inhibiting lysis of erythrocytes.

24. Test kit according to claim 22, wherein the material inhibiting lysis of erythrocytes consists of an erythrocytes aggregating material.

25. Test kit according to claim 22, wherein the material inhibiting lysis of erythrocytes is present in a concentration of 0.2-9 weight percent.

26. Test kit according to claim 22, wherein the erythrocytes stabilising solution further contains a protein digesting enzyme.

27. Test kit according to claim 22, wherein the erythrocytes stabilising solution further contains a preservative.

28. Test kit for the detection of plasma components in blood of human or animal origin, comprising a chromatographic device as described in claim 1, a sample container provided with an aqueous, erythrocytes stabilising solution, a container with an indicator for a plasma component, if desired, a container with an aqueous dilution liquid consisting of an aqueous solution with a pH in the physiological pH-range, and provided with a material inhibiting lysis of erythrocytes, and, if desired, a container with a chase buffer consisting of an aqueous solution with a pH in the physiological pH-range.

29. Test kit for the detection of plasma components in blood of human or animal origin according to claim 28, wherein the erythrocytes stabilising solution is provided with a material inhibiting lysis of erythrocytes.

30. Test kit for the detection of plasma components in blood of human or animal origin according to claim 28, wherein the indicator for a plasma component consists of a gold labelled recombinant protein or gold labelled synthetic peptide.

31. Test kit for the detection of plasma components in blood of human or animal origin according to claim 28, wherein the indicator for a plasma component is included in an aqueous solution.

32. Test kit for the detection of plasma components in blood of human or animal origin according to claim 30, wherein the gold indicator is standardised on an optical density at 540 nm.

32. (canceled)

33. (canceled)

34. A protein comprising a gold labelled EBV protein.

35. Use of gold labelled EBV protein in manufacturing an analysis device for the detection of EBV infection in blood of human origin.

36. Chromatographic analysis device according to claim 2, wherein the dispersant is a poloxamer.

37. Chromatographic analysis device according to claim 3, wherein the circulating cells aggregating material is a hydrocarbon.

38. Chromatographic analysis device according to claim 7, wherein the at least one bivalent transition metal salt is the chloride of calcium.

39. Chromatographic analysis device according to claim 7, wherein the at least one bivalent transition metal salt is the chloride of manganese.

40. Chromatographic analysis device according to claim 7, wherein the at least one bivalent transition metal salt is the chloride of magnesium.

41. Chromatographic analysis device according to claim 8, wherein the bi-functional cross-linking agent is a polyethylene glycol.

42. Chromatographic analysis device according to claim 8, wherein the bi-functional cross-linking agent is glutaraldehyde.

43. Chromatographic analysis device according to claim 8, wherein the bi-functional cross-linking agent is a composition containing a polyethylene glycol and glutaraldehyde.

44. Test kit according to claim 10, wherein the material inhibiting lysis of erythrocytes consists of a dispersant.

45. Test kit according to claim 11, wherein the erythrocytes aggregating material is a hydrocarbon.

46. Test kit according to claim 44, wherein the dispersant is a poloxamer.

47. Test kit according to claim 12, wherein the material inhibiting lysis of erythrocytes is present in a concentration of 0.3-5 weight percent.

48. Test kit according to claim 12, wherein the material inhibiting lysis of erythrocytes is present in a concentration of 1-1.9 weight percent.

49. Test kit according to claim 13, wherein the protein digesting enzyme is a neuromidase.

50. Test kit according to claim 49, wherein the neuromidase is a bromelin.

51. Test kit according to claim 14, wherein the preservative is sodium azide.

52. Test kit according to claim 51, wherein the sodium azide is in a concentration of 0.05%.

53. Chromatographic analysis device according to claim 16, wherein the labelling zone is provided with a gold labelled EBV protein.

54. Chromatographic analysis device according to claim 16, wherein the labelling zone is provided with a gold labelled antibody.

55. Chromatographic analysis device according to claim 17, wherein the detection zones is (are) provided with an antihuman IgM antibody.

56. Chromatographic analysis device according to claim 18, wherein the bi-functional cross-linking agent is a polyethylene glycol.

57. Chromatographic analysis device according to claim 18, wherein the bi-functional cross-linking agent is glutaraldehyde.

58. Chromatographic analysis device according to claim 18, wherein the bi-functional cross-linking agent is a composition containing a polyethylene glycol and glutaraldehyde.

59. Test kit according to claim 22, wherein the material inhibiting lysis of erythrocytes consists of a dispersant.

60. Test kit according to claim 24, wherein the erythrocytes aggregating material is a hydrocarbon.

61. Test kit according to claim 59, wherein the dispersant is a poloxamer.

62. Test kit according to claim 25, wherein the material inhibiting lysis of erythrocytes is present in a concentration of 0.3-5 weight percent.

63. Test kit according to claim 25, wherein the material inhibiting lysis of erythrocytes is present in a concentration of 1-1.9 weight percent.

64. Test kit according to claim 26, wherein the protein digesting enzyme is a neuromidase.

65. Test kit according to claim 64, wherein the neuromidase is a bromelin.

66. Test kit according to claim 27, wherein the preservative is sodium azide.

67. Test kit according to claim 66, wherein the sodium azide is in a concentration of 0.05%.

68. Test kit for the detection of plasma components in blood of human or animal origin according to claim 29, wherein the material inhibiting lysis of erythrocytes is an erythrocytes aggregating material.

69. Test kit for the detection of plasma components in blood of human or animal origin according to claim 29, wherein the material inhibiting lysis of erythrocytes is a dispersant.

70. Test kit for the detection of plasma components in blood of human or animal origin according to claim 30, wherein the indicator for a plasma component is gold labelled EBV protein.

71. Test kit for the detection of plasma components in blood of human or animal origin according to claim 31, wherein the indicator for a plasma component is included in an aqueous solution with a pH in the physiological pH-range.

72. The gold labelled EBV protein according to claim 34, wherein the gold labelled EBV protein is in powder form.

73. The gold labelled EBV protein according to claim 34, wherein the gold labelled EBV protein is in powder form, as a lyophilised powder.
